(19) United States
(12) Patent Application Publication
(10) Pub. No.: US 2018/0020982 A1
(43) Pub. Date: Jan. 25, 2018

(54) WELLNESS MONITORING USING A PATCH SYSTEM

(71) Applicant: Intel Corporation, Santa Clara, CA (US)

(72) Inventors: Adel A. Elsherbini, Chandler, AZ (US); Aleksandar Aleksov, Chandler, AZ (US); Sasha N. Oster, Chandler, AZ (US); Amit Sudhir Baxi, Bangalore (IN); Feras Eid, Chandler, AZ (US); Johanna M. Swan, Scottsdale, AZ (US); Vincent S. Mageshkumar, Navi Mumbai (IN)

(21) Appl. No.: 15/215,529

(22) Filed: Jul. 20, 2016

(51) Int. Cl. A61B 5/00; A61B 5/0205; A61B 5/1455; A61B 5/0408

(52) U.S. Cl. CPC A61B 5/6833; A61B 5/7405; A61B 5/7455; A61B 5/742; A61B 5/746; A61B 5/04087; A61B 5/0205; A61B 5/14551; A61B 5/7271; A61B 5/024

(57) ABSTRACT

Discussed generally herein are methods and devices including or providing a wellness monitoring system. The wellness monitoring system can include a first patch including a flexible, stretchable first substrate, a first adhesive on the first substrate, the first adhesive configured to attach the first patch to skin of a user, and first electronics on or at least partially in the first substrate, the first electronics to monitor a first biological parameter of the user, and a second patch including a flexible, stretchable second substrate, a second adhesive on the second substrate, the second adhesive configured to attach the second patch to skin of the user, and second electronics on or at least partially in the second substrate, the second electronics to monitor a second biological parameter of the user, the second biological parameter different from the first biological parameter.

### TECHNICAL FIELD

This disclosure relates generally to monitoring one or more biologic parameters. One or more embodiments regard using one or more patches to monitor such biologic parameters.

### BACKGROUND ART

Biologic indicators can include resistivity, conductivity, absorption, reflectivity, temperature, moisture content, pressure, motion, expansion/contraction, or capacitance, among others. Biologic indicators can provide an insight into biologic function. Hospitals often monitor biologic function using dedicated machines, such as a heart rate monitor, a blood pressure monitor, or the like. Such monitors can be cumbersome and generally immobile.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A illustrates, by way of example, a perspective view diagram of an embodiment of a patch for monitoring one or more biologic parameters.

FIG. 1B illustrates, by way of example, a perspective view diagram of an embodiment of another patch for monitoring one or more biologic parameters.

FIG. 1C illustrates, by way of example, a perspective view diagram of an embodiment of yet another patch for monitoring one or more biologic parameters.

FIG. 2A illustrates, by way of example, a perspective view diagram of an embodiment of a patch system deployed on a human body.

FIG. 2B illustrates, by way of example, a perspective view diagram of an embodiment of another patch system deployed on a human body.

FIG. 3 illustrates, by way of example, a logical circuit diagram of an embodiment of electronics that can be included in a patch.

FIG. 4 illustrates, by way of example, a perspective view diagram of an embodiment of a stretch sensor.

FIG. 5 illustrates, by way of example, a perspective view diagram of an embodiment of ECG electrodes.

FIG. 6 illustrates, by way of example, a logical block diagram of an embodiment of a patch network.

FIG. 7 illustrates, by way of example, a logical block diagram of an embodiment of another patch network.

FIG. 8 illustrates, by way of example, a logical block diagram of an embodiment of yet another patch network.

FIG. 9 illustrates, by way of example, a logical block diagram example of a system which includes items that can be included in the electronics of a patch.

### DESCRIPTION OF EMBODIMENTS

The following description and the drawings sufficiently illustrate embodiments to enable those skilled in the art to practice them. Other embodiments can incorporate structural, logical, electrical, process, or other changes. Portions and features of some embodiments can be included in, or substituted for, those of other embodiments. Embodiments set forth in the claims encompass all available equivalents of those claims.

Embodiments discussed herein regard monitoring one or more biologic parameters (e.g., one or more biologic indicators, biologic functions, and/or biologic conditions) using multiple patches. Generally, multiple patches can be deployed on a user. The multiple patches can each monitor a same or different biologic parameter. This data from the patches can be analyzed to determine a wellness state of an entity wearing the patches.

Continuous monitoring of vital signs can be important in many applications. For example, in hospitals, continuous monitoring of heart rate is quite common for many, if not most, patients. In sports, continuous monitoring of heart rate, breathing rate, EMG (electromyography), speed, and/or range of motion can help in evaluating an athlete's flexibility, cardiovascular conditions, and weakness. Other applications, for example, can include posture detection (e.g., in office environments to avoid ergonomic problems that can lead to a condition which makes the employee unfit for work or in video game programming to create realistic motion of three dimensional models) or physical therapy (e.g., to detect an improvement in the range of motion and/or motor skills).

Some approaches have been proposed for continuous monitoring of vital signs. Each has their own drawbacks. For continuous monitoring of heart rate, hospitals connect ECG (electrocardiogram) probes to the patient which then connect to bulky ECG monitors. In sports, smart watches/bands and chest straps are commonly used to monitor heart rate. In calorie monitoring, the smart watches/bands depend on statistical modeling to estimate the calories burned and the motion involved, since they cannot collect information about the different moving joints in the athletes body. The patch systems can improve upon the calorie estimation by collecting information about joint movement, torso movement, flexion, extension, and the like, to provide a more accurate estimate on the amount of calories burned. Other approaches use stretchy clothes with embedded probes which can impact the athletic performance and can be uncomfortable to wear for extended periods.

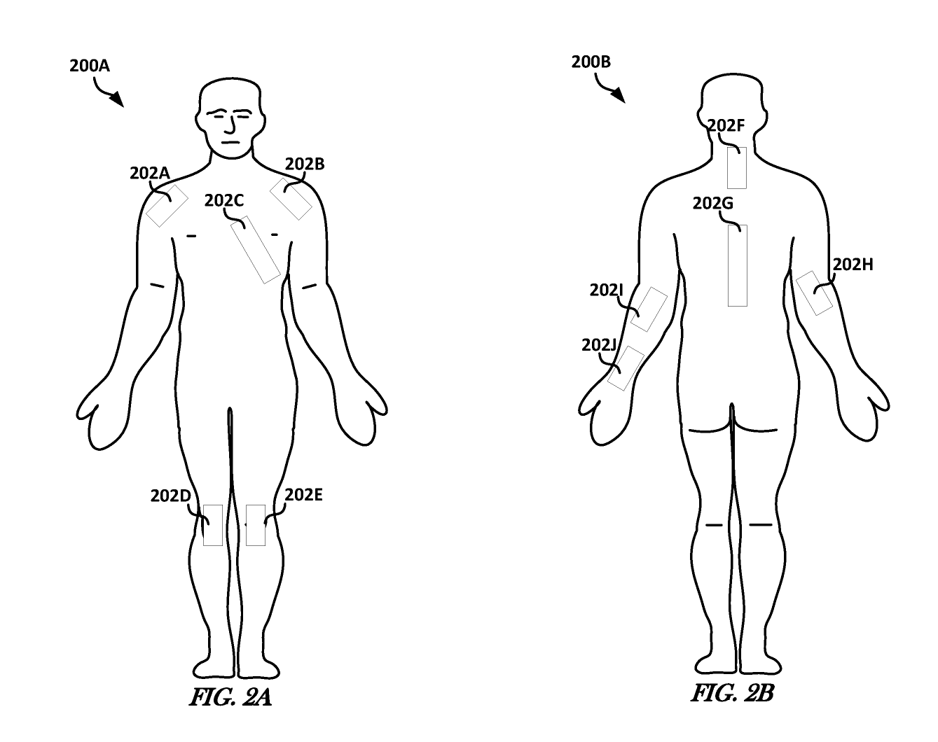

Discussed herein are stretchable fabrics/elastomers with integrated electronics that can be attached to the skin, such as through an adhesive. Several capabilities can be integrated into one patch or singular capabilities can be integrated into multiple patches. Two or more of those patches can then be attached to different areas on the body for different monitoring, such as shown in FIGS. 2A and 2B.

The adhesive patch can include a stretchable elastomer with a stretchable adhesive and electronics situated thereon or at least partially therein. The patch can help keep the electronics in close contact with skin of a user. The electronics can either be a rigid block, flexible block, or multiple rigid and/or flexible blocks.

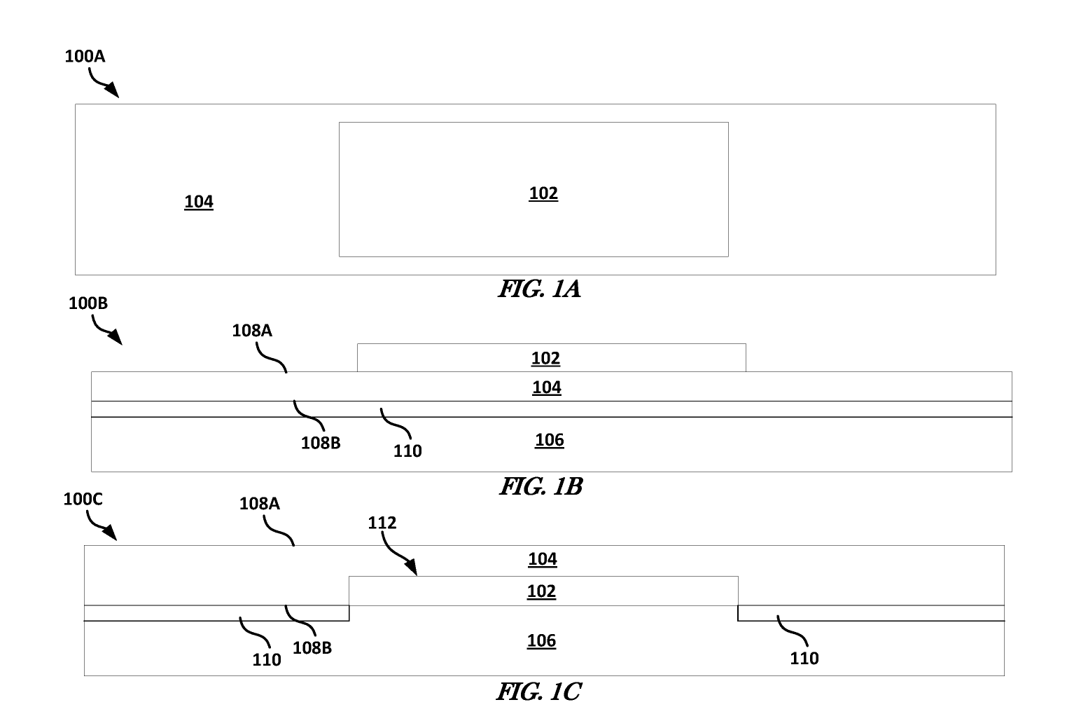

Several variations are possible for the adhesive patch, for example, the adhesive patch can include conductive traces to probe the skin (e.g., for ECG signals) or to connect multiple electronic blocks together. The adhesive patch can also include conductive/electrically functional traces that can provide electrical functionality (e.g., form an antenna or measure strain, temperature, stretch, or the like). The patch and the electronics are illustrated as including a rectangular footprint in FIGS. 1A, 1B, and 1C, but the shape can be adjusted according to the application (e.g., elliptical, polygonal, T-shape, H-shape etc.).

The patches discussed herein can have one or more advantages over hospital bedside equipment and/or other wellness monitoring device. Such advantages can include one or more of: (1) Enabling patient mobility allowing the patient to move without impacting the signal or disconnecting the probe wires; (2) Providing better hygiene. The portion contacting the skin can disposable and the electronics can be packaged in a way to make them easily sanitized; and/or (3) Can monitor more than one biologic parameter on a single patch or through multiple patches. Other biologic parameters or indicators that can be monitored include blood pressure or other pressure, skin temperature, breathing rate, and/or muscle movements, among others. Additionally or alternatively, security features can be implemented to allow physicians to activate the electronics when they are attached and/or the automatically deactivate (power off) a patch when it is detached (e.g. using proximity sensor, ECG pads, and or a strain sensor).

In sports, physical therapy and game design applications, the patch systems discussed herein can provide one or more of the following advantages: (1) The patches are in intimate contact with the skin, allowing accurate monitoring of the position and/or stretch movement; (2) The patches are light weight and will have minimal impact on the user compared to stretch clothing or bulkier devices; (3) The patches can have relatively low Z-height (i.e. thickness as defined as a distance from a surface of the skin away from the body of a user on a line tangent to the surface of the skin) enabling them to be worn discreetly and/or in a non-invasive manner; and/or (4) The patches can be easily customizable allowing them to fit different body types and sizes and/or monitor different biologic parameters.

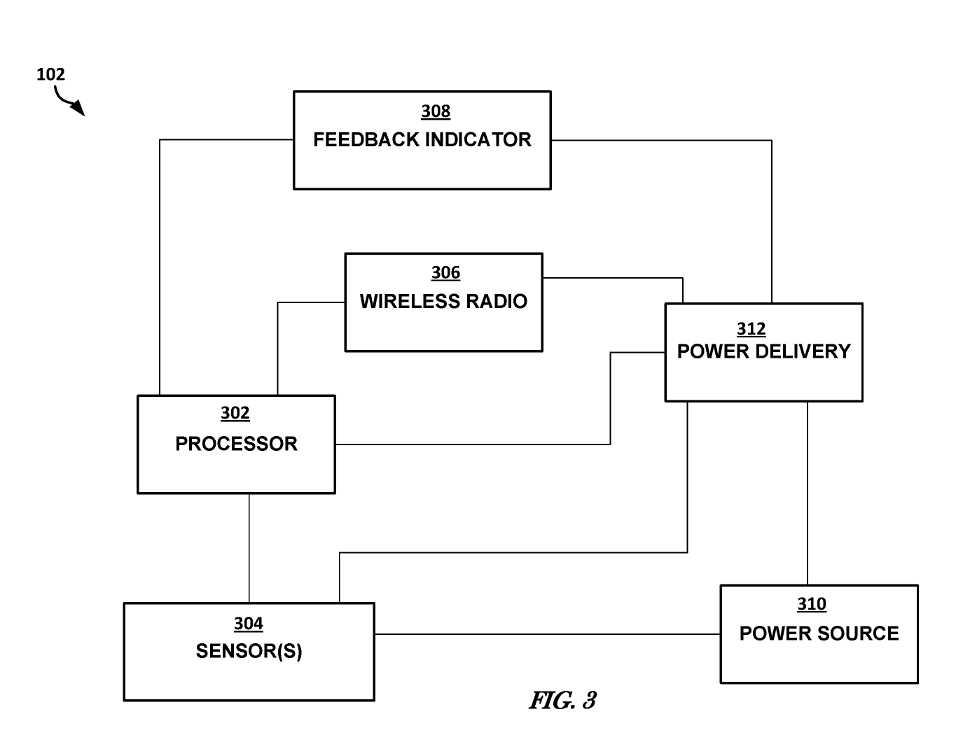

FIG. 1A illustrates, by way of example, a perspective view diagram of an embodiment of a patch 100A. The patch 100A as illustrated includes electronics 102 and a substrate 104. The electronics 102 can include one or more of the items as discussed with regard to FIGS. 3, 4, and 5. The electronics 102 can include one or more electric or electronic components, such as can include a resistor, capacitor, transistor, inductor, diode, regulator, sensor (e.g., a temperature sensor, oxygen sensor, stretch sensor, inertial measurement unit (IMU) sensor, and/or electrocardiogram (ECG) sensor, Electromyography (EMG) sensor, Audio sensor (e.g., microphone could potentially detect heart beat), pressure sensor (e.g., piezo sensor could pick up pulse, heartbeat, fetal movement, etc.), ultrasonic sensor (paired with ultrasound emitter could be used for imaging, heartbeat, fetal features, etc.); chemical sensors such as a salinity or a lactic acid sensor, optical sensors (to obtain information about levels of certain enzymes/chemicals in blood), among others), accelerometer, optical component (e.g., a light emitting diode (LED)), multiplexer, processor, memory, battery, antenna, modulator/demodulator, radio (e.g., receive or transmit radio or a transceiver), and/or amplifier, or the like. The electronics 102 are arranged to monitor a biologic parameter of the entity wearing the patch. The electronics 102 can include a single flexible or rigid block or multiple flexible or rigid blocks, such as to allow for more conformal coverage of the body.

The substrate 104 can include a stretchable and/or flexible material, such as can include an elastomer, spandex, woven fabric, plastic (e.g., polyvinyl chloride (PVC), polyethylene, and/or polyurethane), TPU (thermoplastic polyethylene), polydimethylsiloxane (PDMS) (silicone), latex, or a combination thereof, among others. As used herein “stretchable” and “flexible” are different. Stretchable connotes an ability to lengthen and flexible connotes an ability to rotate and/or bend without significant elongation.

FIG. 1B illustrates, by way of example, a perspective view diagram of an embodiment of another patch 100B. The patch 100B as illustrated includes the electronics 102 on a top surface 108A of the substrate 104. The patch 100B as illustrated includes an adhesive 110 on a bottom surface 108B of the substrate 104. The bottom surface 108B is opposite the top surface 108A. The bottom surface 108B faces skin 106 of a user. The top surface 108A faces away from the skin 106 of the user. The substrate 104 can be optically transparent, such as to allow light from the electronics 102 to be incident on the skin 106.

The adhesive 110 can at least temporarily affix the patch 100B to the skin 106. The adhesive 110 can include a silicone (polydimethylsiloxane (PDMS)) adhesive, double-sided tape or an acrylate (e.g., methacrylate or epoxy diacrylate, among others), among others.

FIG. 1C illustrates, by way of example, a perspective view diagram of an embodiment of yet another patch 100C. The patch 100C is similar to the patch 100B with the patch 100C including a cavity 112 in the substrate 104. The electronics 102 are situated, at least partially, in the cavity 112. The adhesive 110, as illustrated, is located on portions of the substrate 104 around the electronics 102. In one or more embodiments, the adhesive 110 can be located on the electronics 102.

More details regarding the patch 100A-C, electronics 102, and substrate 104, including how to make the patch 100A-C, can be found in Patent Cooperation Treaty Patent Application PCT/US2016/040476, titled “Devices and Methods for Sensing Biologic Function”, and filed on Jun. 30, 2016, which is incorporated herein by reference in its entirety.

FIG. 2A illustrates, by way of example, a perspective view diagram of a body with patches 202A, 202B, 202C, 202D, and 202E in various locations thereon. The patches 202A-E can be similar to the patches 100A-C.

The patch 202A is located on a front side of a right shoulder. The patch 202B is located on a front side of a left shoulder. The patch 202A-B can include electronics to monitor flexion, contraction, and/or stretch (e.g., using a stretch sensor), temperature, electrical activity (e.g., using an EMG), and/or a specific force, angular rate, and/or magnetic field (e.g., using an inertial measurement unit (IMU)), among others.

An IMU is a device that measures a specific force, angular rate, and/or magnetic field using one or more accelerometers, gyroscopes, and/or magnetometers. An EMG can help assess of muscles and/or nerve cells that cause the muscles to contract or extend. The EMG translates the electrical signals from the nerve cells electrical signals that are processed and can be used to generate a report (e.g., sound, numerical value, and/or graph) that can be interpreted by a specialist to determine the electrical activity produced by/for muscles or by the user (e.g., to help ensure safe exercise or correct posture). The stretch can be interpreted to indicate an amount contraction or flexion of a muscle, a respiration rate, and/or a rotation (e.g., of the torso or other part of the body). The temperature can be monitored using a temperature detector, such as a temperature resistance detector (TRD). The TRD has a resistance that varies with temperature. Measuring the resistance of the TRD can thus indicate a temperature of an environment around the TRD.

The patch 202C is located on a bust. The patch 202C can include electronics to monitor electrical and/or muscular activity of a heart (e.g., using ECG probes), stretch, organ or organ or conduit (e.g., vein or artery, among others) volume (e.g., using a photoplethysmogram (PPG), such as a pulse oximeter), blood or other pressure, temperature, respiration, salinity, oxygen (e.g., via PPG), a specific force, angular rate, and/or magnetic field (e.g., using an IMU), and/or others. A combination of the ECG and PPG can be used to estimate the blood pressure as is described in Patent Cooperation Treaty Application Patent Cooperation Treaty Patent Application PCT/US2016/040476, referenced supra. Salinity can be measured using a salinity sensor. A salinity sensor measures an amount of dissolved salt in an aqueous solution.

The patch 202D is located on a right knee. The patch 202E is located on a left knee. The patch 202E can include electronics to measure flexibility (e.g., using a stretch sensor, such as shown in FIG. 3), stretch, temperature, electrical activity (e.g., using an EMG), and/or specific force, angular rate, and/or magnetic field (e.g., using an IMU), among others.

FIG. 2B illustrates, by way of example, a perspective view diagram of a body with patches 202F, 202G, 202H, 202I, and 202J in various locations thereon. The patches 202F-J can be similar to the patches 100A-C or the patches 202A-E.

The patch 202F is located along a spine on a neck. The patch 202F can include electronics to monitor muscular and/or electrical activity of the heart (e.g., using an ECG), temperature, stretch, pressure (via piezoelectric materials), organ or organ or conduit (e.g., vein or artery, among others) volume (e.g., using a photoplethysmogram (PPG), such as a pulse oximeter), blood pressure (e.g., using a combination of the ECG and PPG), electrical activity, and/or a specific force, angular rate, and/or magnetic field (e.g., using an IMU), among others.

The patch 202G is located along a spine on a back. The patch 202C can include electronics to monitor stretch, temperature, electrical activity (e.g., using an EMG), posture, and/or a specific force, angular rate, and/or magnetic field (e.g., using an IMU), among others.

The patch 202H is located on a right elbow. The patch 202I is located on a left elbow. The patch 202H can include electronics to measure flexibility and/or posture (e.g., a stretch sensor, such as shown in FIG. 3), stretch, temperature, electrical activity (e.g., using an EMG), and/or a specific force, angular rate, and/or magnetic field (e.g., using an IMU), among others.

The patch 202J is located on a wrist. The patch 202J can include electronics to monitor stretch, temperature, electrical activity (e.g., using an EMG), and/or a specific force, angular rate, and/or magnetic field (e.g., using an IMU), among others.

In one or more embodiments, the patch 100A-C or 202A-J can include one or more of: (1) Stretchable substrate with adhesive, providing mechanical attach functionality and/or an electrically conductive path to the skin; (2) Stretchable substrate with conductive traces enabling electrical functionality (e.g., antenna or stretch sensor); (3) Stretchable substrate with conductive traces enabling electrical contact to the skin (e.g., ECG pads or electro-dermal activity traces (e.g., to measure galvanic skin response, skin conductance, psychogalvanic reflex, skin conductance response, skin conductance response, sympathetic skin response, and/or electro-dermal response); (4) Stretchable substrate with attached LEDs, haptics, or other indicators to provide visual feedback to the user or the observer (e.g., heart rate or posture feedback).

In one or more embodiments, the electronics 102 can include: (1) Energy storage (battery or super capacitor) and/or energy harvesting (e.g., solar, RF or temperature based); (2) Data recording functionality (e.g., for skin galvanometer or stretch sensor); (3) Wireless or wired data transfer (for transferring to cell phone or another device) (e.g., Bluetooth, Near field communication (NFC), and/or Cellular, among others); (4) Functional blocks (e.g., one or more IMUs (can be used for data correction or for information on the user)) or temperature sensor (for body temperature sensor) or other types of biosensors (e.g., sensors previously discussed); and/or (5) One or more indicators, such as an LED, or integrated display for real time monitoring of the user's state. Indicators can also be acoustic (e.g., one or more speakers) or haptic feedback (e.g., motors).

FIG. 3 illustrates, by way of example, a functional block diagram of an embodiment of the electronics 102. The electronics 102 as illustrated include a processor 302, sensor(s) 304, a wireless radio 306, a feedback indicator 308, a power source 310, and power delivery circuitry 312. The electronics 102 may include one or more of the items illustrated in FIG. 3.

The processor 302 includes electric or electronic components arranged to perform operations on signals received at the processor 302. The processor 302 can include an application specific integrated circuit (ASIC), field programmable gate array (FPGA), or the like. The processor 302 can send and receive signals to/from the sensor(s) 304, the wireless radio 306, and the power delivery circuitry 312. The processor 302 can include an analog to digital converter (ADC). The power delivery circuitry 312 can provide the processor 302 with the electrical power required for the operation of the processor 302.

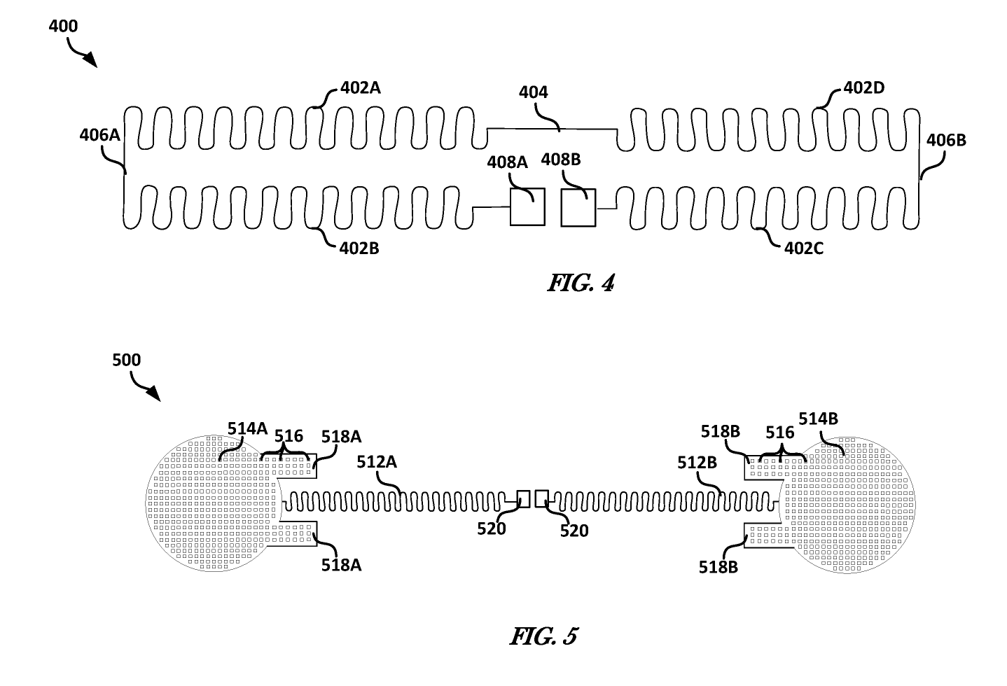

The sensor(s) 304 can include one or more of an ECG electrode (see FIG. 5), electro-dermal activity sensor, a stretch sensor (see FIG. 4), a photo diode (or other light detector), a temperature detector, a salinity sensor, a power level sensor, a pulse oximeter, and/or a resistivity sensor, among others, such as can include one or more sensors discussed herein. One or more of the sensor(s) 304 may require electrical power to operate, such power can be provided directly from the power source 310 or from the power delivery circuitry 312.

The wireless radio 306 can include a receiver, transmitter, transceiver, antenna, modulator, demodulator, amplifier, and/or other circuitry associated with receiving and/or transmitting electrical signals in a wireless manner. The wireless radio 306 can provide signals received to the processor 302. The wireless radio 306 can transmit signals received from the processor 302. The wireless radio 306 can be powered directly by the power source 310 or the power delivery circuitry 312.

The feedback indicator 308 can include an audio, visual, and/or tactile feedback component. An audio feedback component can include a speaker and associated signal conditioning circuitry, such as an amplifier. A visual feedback component can include a display, such as a touch screen display, LED display, liquid crystal display (LCD), or the like. A tactile feedback component can include a motor, a fan, a heat generating component, or the like.

The power source 310 can include a dry cell battery, a super capacitor, a solar panel, an inductive coupler, or the like. The power source 310 can provide electrical power directly to any of the processor 302, the sensor(s) 304, the wireless radio 306, the feedback indicator 308, and/or the power delivery circuit 312. Not all items of the FIG. 3 are illustrated as being connected to the power source 310 so as to not obscure the view in the FIG. However, any of the items in FIG. 3 may be directly connected or otherwise coupled to the power source 310.

The power delivery circuitry 312 can receive power from the power source 310 and modify one or more of the current and voltage of the power to be compliant with a power requirement of a component to receive the power. The power delivery circuitry 312 can include one or more of a voltage regulator, current regulator, current source, voltage booster, current booster, amplifier, or the like to alter a current or voltage of the electrical power from the power source 310.

The processor 302 can receive signals from the sensor(s) 304 and perform operations based on the state of the signals received. For example, the processor 302 can receive a signal from one of the sensor(s) 304 and compare a value corresponding to the signal, to one or more threshold values. If the value is less than (or equal to), greater than (or equal to), or a combination thereof, one or more of the threshold values, the processor 302 can perform an operation in response thereto. The operation can include providing a signal to the feedback indicator 308 that causes the feedback indicator 308 to indicate that the monitored value is in or out of range. In another example, the processor 302 can perform operations on signals received from one or more of the sensor(s) 304 to determine a value associated with a biologic parameter. For example, the processor 302 can receive signals from an ECG electrode and a photo detector and determine an estimate of blood pressure based on those signals. In another example, the processor 302 can receive signals from a stretch sensor (see FIG. 4) and determine a respiration rate based on those receive signals.

FIG. 4 illustrates, by way of example, a perspective view diagram of an embodiment of a stretch sensor 400. The stretch sensor 400 as illustrated includes a plurality of sections of meandering traces 402A, 402B, 402C, and 402D, conductive connectors 404, 406A, and 406B, and contact pads 408A and 408B. The meandering traces 402A and 402D are electrically connected through the conductive connector 404. The meandering traces 402A-B are electrically connected through the conductive connector 406A. The meandering traces 402C-D are electrically connected through the conductive connector 406B. The meandering trace 402B is electrically connected to the contact pad 408A. The meandering trace 402C is electrically connected to the contact pad 408B.

As the substrate 104 stretches or flexes, the meandering traces 402A-D will stretch or flex. As the meandering traces 402A-D stretch or flex, the resistivity and/or conductivity of the meandering traces 402A-D changes. A signal can be provided to either of the pads 408A-B and a return signal can be provided on the other of the pads 408A-B. The change in an electrical property (e.g., voltage, current, resistance, or the like) between the provided signal and the return signal indicates a stretch state of the stretch sensor 400. The fluctuations in the electrical property can indicate, for example, a respiration rate.

FIG. 5 illustrates, by way of example, a perspective view diagram of an embodiment of ECG electrodes 500. The ECG electrodes 500 are dry conductive electrodes, as compared to conventional ECG electrodes with conductive gel (that can dry out within a few hours). Using conductive gel, the ECG signal from the ECG electrodes progressively deteriorates. The ECG electrodes 500 can include perforations 518 therein. The perforations 518 are voids in the electrodes 500. The perforations 518 can aid in anchoring the electrodes 500 to the substrate 104. The perforations 518 can help reduce overall cost of the device, such as when using an additive manufacturing technique (e.g., conductive ink printing). The perforations 518 can help increase flexibility without significantly impacting performance (e.g., electrical performance characteristics) of the electrode 500.

The traces 512 electrically connect the electrodes 510 to respective pads 520. The traces 512 can be formed with a meandering pattern, such as to increase the stretchability and/or flexibility of the patch 100A-C and 202A-J as compared to using generally straight traces. If stretchable conductive material is used (e.g., stretchable conductive ink), the peak to peak amplitudes of signals on the traces 512 can be reduced.

The electrodes 500 as illustrated include a plurality of stretch limiting patches 518A and 518B connected thereto. The stretch limiting patches 518A-B can include metallization with perforations 516. The stretch limiting patches 518A-B are positioned to help prevent breakage of other metallization, such as a junction between a main body 514A-B of the electrodes 500 and the traces 512. The stretch limiting patches 518A-B constrain the strain in the regions in and around the stretch limiting patches and help prevent over-stretching in areas that might otherwise break if the patch 100A-C or 202A-J is stretched. The ECG electrodes 500 can be low-profile, thin, stretchable, flexible, low-cost, and/or disposable.

Use Case: Athletic Performance Evaluation

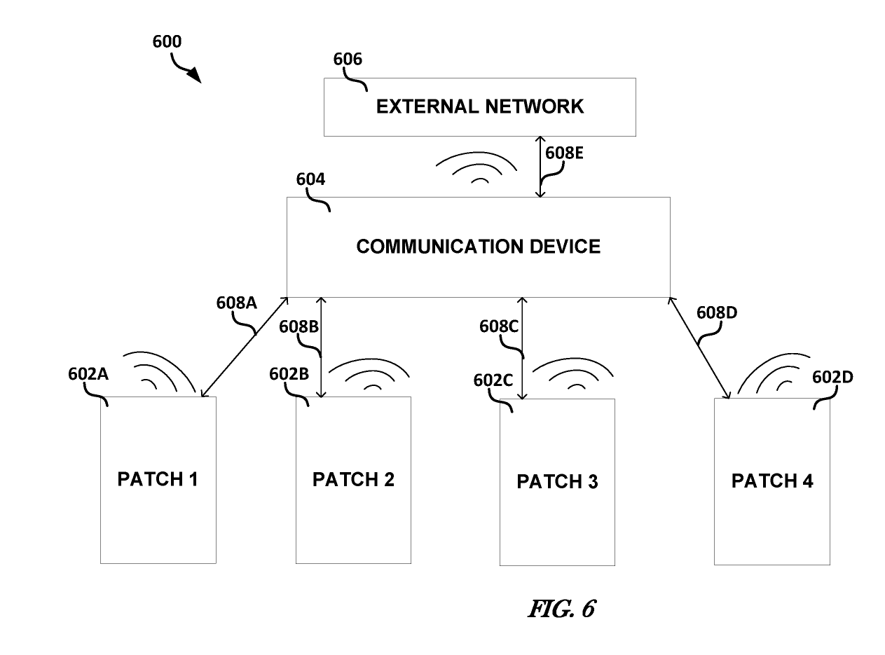

Patches can be located at the joints of the body that are performing, such as for continuous monitoring for the particular sports (e.g., legs for running, elbows and shoulders for rowing, legs and arms for swimmers, etc.). Depending on the nature of the sport and use conditions, the patches can either form a body area network to communicate all the information to a central patch or to phone/other wearable device (see FIGS. 6-8). An alternative lower cost and lower power option is for the patches to store the information internally then the information are downloaded later through direct electrical connection or through NFC. In such embodiments, the electronics 102 can include a memory connected to the processor 302.

Use Case: Posture Monitor

Long periods of continuous computer use, which occurs in many office environments without proper posture, can lead to chronic back pain and even spinal injuries. It can be helpful in these cases to provide feedback (e.g., relatively continuous feedback) to users, such as to help prevent injury. A posture monitor can include a single longer patch, and may be mounted on the user's back with one or more stretch sensors on the stretchable portion, and an inertial measurement unit (IMU) sensor in the electronics 102. Such a patch can monitor the user's spine arch and provide haptic, visual, or audio feedback (through the feedback indicator 308) when the user's posture is incorrect, such as for a specified period of time. In one or more embodiments, such as patch can communicate with the user's phone, a wearable device, or other communication device (such as through the wireless radio 306) to provide feedback or for recording the information for later analysis.

Use Case: Physical Therapy and Rehab

After some injuries, such as bone fractures or joint dislocations, physical therapy can be helpful to restore joint mobility and achieve certain range of motion. In many such situations, kinesiology tape can be used to help with the healing and/or for pain relief. The same kinesiology tape can provide the substrate (e.g., the substrate 104) and can have electronics that can include an integrated stretch sensor and a data recorder (i.e. a processor and memory). The data recorder can record the stretch or just data signals from the stretch sensor over time. Such data signals can be used to monitor the progress and provide additional information to the doctors about each patient's progress. This patch can be applied at different joint locations and depending on the power budget, can either send the data using cellular network, to a cell phone or recorded for later evaluation by the therapist.

FIG. 6 illustrates, by way of example, a logical block diagram of an embodiment of a system 600 for monitoring one or more biological parameters. In the system 600, each patch 602A, 602B, 602C, and 602D communicates signals directly to an intermediate communications device 604, such as a cell phone or tablet. Each patch 602A-D has the ability to communicate with a network 606, such as through the external device 604, but does not have to communicate with other patches 602A-D. In such embodiments, either the network 606 or the external device 604 performs analysis and/or synthesis of data from multiple patches 602A-D. The analysis and/or synthesis can include determining, based on data from two or more patches, whether a medication dosage is too high or too low, whether a therapy (e.g., an ultrasonic or electro-therapy) should be delivered, whether another sensor measurement should be performed by one or more of the patches 602A-D, or the like.

Each of the electronics of the patches 602A-D includes a transceiver and associated circuitry to allow for wireless communication between the external device 604 and the patches 602A-D. The integration of the patches 602A-D into a body area network (and sensor data analysis/synthesis) happens entirely virtual, such as in the network 606 and/or the device 604. In the system 600, each patch 602A-D acts independently of the other patches 602A-D. Electronics of the external device 604 can include a transceiver to receive data from the patches 602A-D and transmit data to the patches 602A-D. The transceiver of the external device can receive data from and transmit data to the network 606. The network 606 can be a network accessible by a nurse, doctor, physician's assistant, or other qualified medical personnel. The data from the communication device 604 to the network 606 can be stored for subsequent analysis by personnel with access to the network 606. The communication device 604, in one or more embodiments, can perform one or more operations on data received. The device 604 can compare the data (before or after the operations) to a specified range of values, such as to determine if an alert is to be sent (by the transceiver) to one or more of the patches 602A-D that includes a feedback indicator and/or to the network 606. In one or more embodiments, the device 604 can provide the alert to the user, such as through a feedback indicator of the electronics of the device 604.

Arrows 608A, 608B, 608C, 608D, and 608E indicate possible directions for data flow using the system 600. While bi-directional communication between each of the patches 608A-D and the device 604 is possible, one or more embodiments may include only uni-directional communication, such as from one or more of the patches 602A-D to the communication device 604 and not vice versa. In such embodiments, electronics of the patch 602A-D may only include a transmit radio (and not a receive radio) and associated circuitry for transmitting data. Consider an embodiment in which a patch is collecting ECG data. This data may be relayed to the device 604 without the device 604 communicating to the patch.

In one or more embodiments, data analysis/synthesis can be performed, at least partially, by electronics of the patch 602A-D. The data before and/or after the synthesis can be communicated to the device 604.

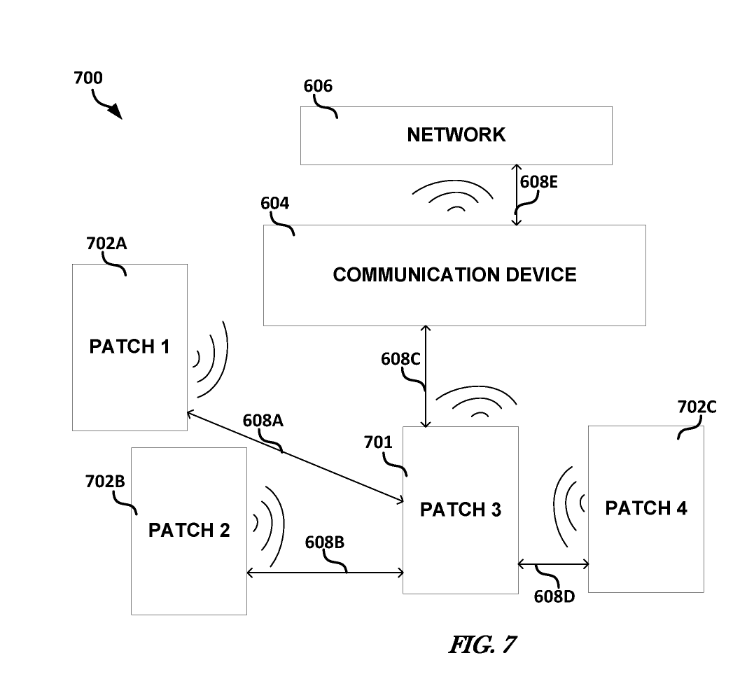

FIG. 7 illustrates, by way of example, a logical block diagram of another embodiment of a system 700 for monitoring one or more biological parameters. In the system 700, each patch 702A, 702B, and 702C communicates signals directly to a master patch 701. The master patch 701 is the only patch that communicates with the intermediate communications device 604. Each patch 702A-C has the ability to communicate with the network, such as through the master patch 701 and the external device 604, but does not communicate directly with the device 604. In such embodiments, the network 606, the master patch 701, and/or the external device 604 performs analysis and/or synthesis of data from multiple patches 702A-C.

The master patch 701 can include a battery (or other power source) with a larger capacity than batteries (or other power sources) of the patches 702A-C. The master patch 701 can be configured to receive signals from the patches 702A-C that are time domain multiplexed, frequency domain multiplexed, or other scheme that provides an indication to the master patch 701 which patch 702A-C a given transmission was from. Time domain multiplexing provides the patches 702A-C with different, non-overlapping time slots to communicate with the master patch 701. Frequency domain multiplexing provides the patches 702A-C with different frequency channels over which to communicate with the master patch 701.

In one or more embodiments, the patches 702A-C can provide data to the master patch 701 in response to a memory of the electronics of the respective patch 702A-C including a specified amount of data stored thereon, a specified amount of time elapsing, determining that data from a sensor of the electronics indicates a biological parameter is in or out of a specified range of values, receiving a communication from the master patch 701 indicating that the patch 702A-C can send the data, or the like.

The master patch 701 is responsible for all communication outside the body area network (i.e. the patches). The master patch 701 can, like the patches 702A-C, monitor, using electronics, one or more biological parameters. The master patch 701 can perform analysis/synthesis of data from the electronics of the master patch 701 and/or one or more of the patches 702A-C.

The master patch 701 is the only patch that communicates with the intermediate communications device 604. Each patch 702A-C has the ability to communicate with the network, such as through the master patch 701 and the external device 604, but does not communicate directly with the device 604. In such embodiments, the network 606, the master patch 701, and/or the external device 604 performs analysis and/or synthesis of data from multiple patches 702A-C.

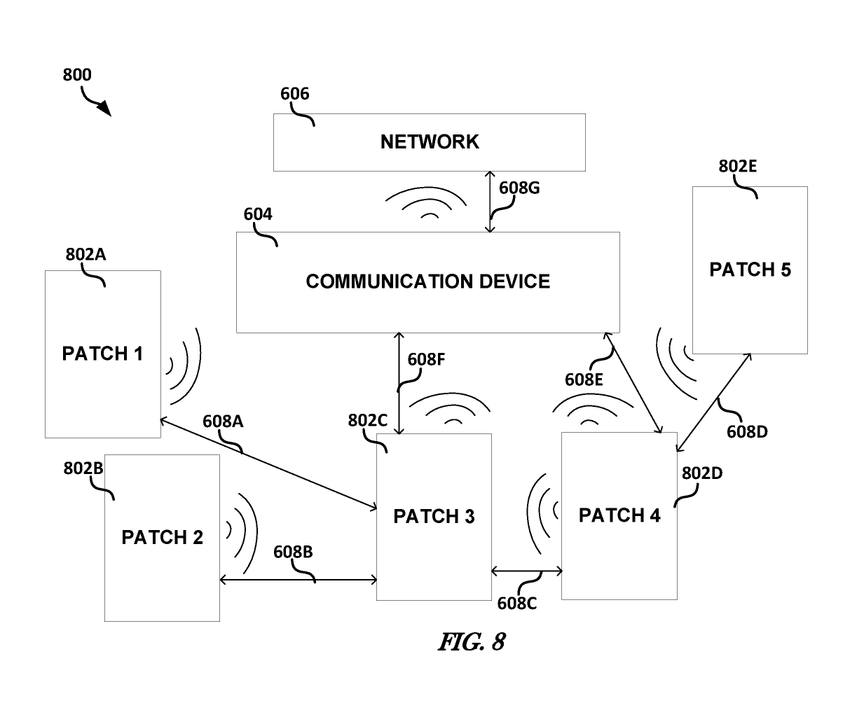

FIG. 8 illustrates, by way of example, a logical block diagram of another embodiment of a system 800 for monitoring one or more biological parameters. In the system 800, each patch 802A, 802B, 802C, 802D, and 802E can take a role of a master patch or a slave patch. One patch 802C can take the role of the master patch at a first time and another patch 802D can take the role of the master patch at a different time. The master patch 802C-D at a given time is the only patch that communicates to the device 604. The slave patches (any patch 802A-E that is not the master patch at a given time) communicate to the master patch and not with the device 604.

In one or more embodiments, a patch performs a role of a hybrid master-slave. A hybrid master-slave receives data from another patch and forwards the data to a master patch, but does not communicate directly with the device 604 when the hybrid master-slave is not performing a master role. For example, when the patch 802C is performing the role of the master, the patch 802E can provide data to the patch 802D and the patch 802D can provide the data from both patches 802D and 802E to the patch 802C. The patch 802C can communicate data from all the patches 802A-E to the device 604, such as simultaneously (in a same packet) or in a time multiplexed or frequency multiplexed manner.

The patch that acts as a master can be determined based on a heuristic or a pre-defined schedule. The heuristic can be based on available power at the patch, current processing load of the patch, power usage of the patch, proximity to the device 604, and/or range of a wireless radio of the patch, among others. Data indicating which patch is determined to be a master can be communicated, such as by a current master patch, to remaining patches. The patch can then act as master, such as for a certain period of time, and the heuristic can be re-computed for each of the patches. The current master can then remain the master or a new master can be declared and the new master can act as a master, such as for a specified period of time. Such embodiments can provide for a more even power-use distribution among the power sources of the patches. There can be slave patches that remain slaves as they have no capability to radio to outside of the body network, but to limit power, for example, all communication these patches are capable of is within the body-area network. Thus, FIG. 8 illustrates a block diagram of a body-area multi-patch network with dynamic and autonomous patch assignments. Slave patches are patches that do not communicate to the outside world either due to assignment or if they were designed only to work within a body-area network. A master patch is the patch (or the patches) responsible for communication with the outside (of the body-area) network.

Communication between patches is an analogue to communication between any two or more computational devices. Sensor fusion and data transfer to and from the cloud is also a reality. Wireless radios for low power exist and components of the above mentioned sensors and electronics exist. These concepts and items are used in a system, as it would be any wireless networked system, however this one based on flexible and stretchable systems on the human body to provide functionality to monitor biological parameters of an entity wherein the patches. Such body area networks provide an ability to provide unique functionality and/or provide one or more advantages over current systems and methods for monitoring such biological parameters.

The data of the body-area multi-patch system (i.e. the data collected and/or processed by the patches of the body-area system) can be analyzed by a computing system capable of sensor-fusion, machine learning, and/or connected to a database of physiological signals and responses to correctly analyze the state of the patient and formulate a response. All these systems can include components including CPU's, co-processors, and/or wireless radios, among other electronics.

In one or more embodiments, one or more of the patches can communicate directly with one or more other patches of the body-area network, such as for better response to biological parameters. For example, an increase of perspiration not related to increased muscle activity or heart rate can be an alarming sign that can be detected when the sensors of the multi patch system work together to allow for the synthesis of an overall body view that cannot be captured by a single patch. In another example, if an increase in heart rate (e.g., not tachycardia or other arrhythmia) is not associated with increased muscular activity, motion, or breathing this can also be a sign for concern. Fluctuations in blood pressure not associated with changes in heart rate can be associated with problems of the circulatory system other than the heart. An increase in pain related chemicals in the sweat can be checked against the posture of the patient either indicating bad posture leading to pain or the pain leading to bad posture to counteract it (depending on the patient situation). This combined sensory input can then lead to a more comprehensive and corrective therapeutic input of personnel (e.g., medical professional) monitoring the patient.

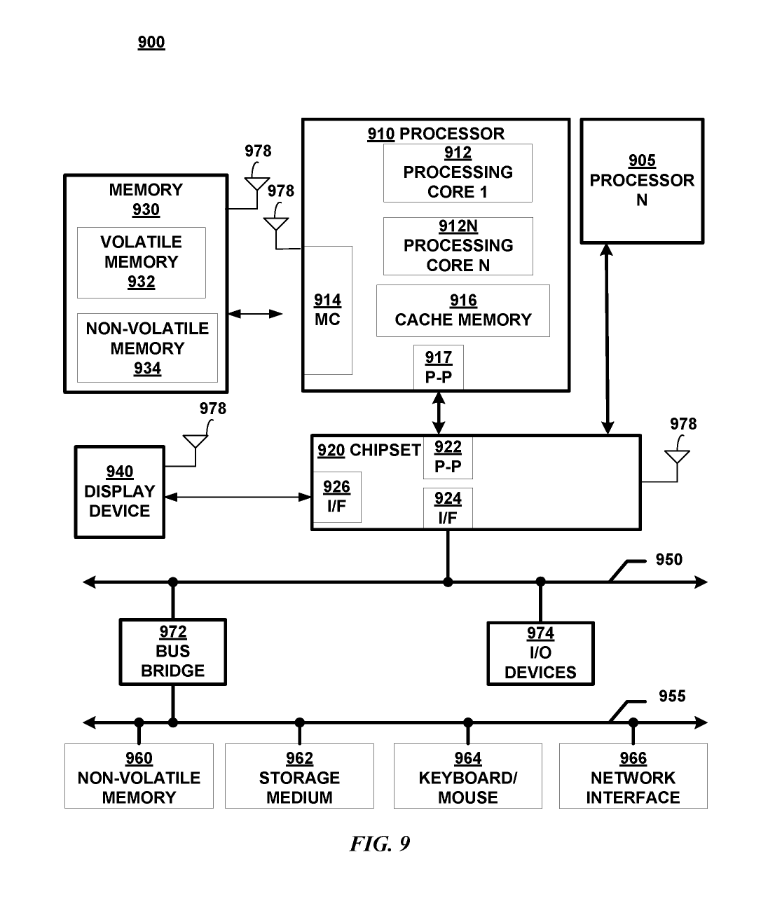

FIG. 9 illustrates, by way of example, a logical block diagram of an embodiment of an system 900 that includes components which can be included as part of the patch 100A-C and/or 202A-J. The patch 100A-C and/or 202A-J can include one or more of the items of the system 900, such as part of or connected to the electronics 102.

In one embodiment, processor 910 has one or more processing cores 912 and 912N, where 912N represents the Nth processor core inside processor 910 where N is a positive integer. In one embodiment, system 900 includes multiple processors including 910 and 905, where processor 905 has logic similar or identical to the logic of processor 910. In some embodiments, processing core 912 includes, but is not limited to, pre-fetch logic to fetch instructions, decode logic to decode the instructions, execution logic to execute instructions and the like. In some embodiments, processor 910 has a cache memory 916 to cache instructions and/or data for system 900. Cache memory 916 may be organized into a hierarchal structure including one or more levels of cache memory.

In some embodiments, processor 910 includes a memory controller 914, which is operable to perform functions that enable the processor 910 to access and communicate with memory 930 that includes a volatile memory 932 and/or a non-volatile memory 934. In some embodiments, processor 910 is coupled with memory 930 and chipset 920. Processor 910 may also be coupled to a wireless antenna 978 to communicate with any device configured to transmit and/or receive wireless signals. In one embodiment, the wireless antenna interface 978 operates in accordance with, but is not limited to, the IEEE 802.11 standard and its related family, Home Plug AV (HPAV), Ultra Wide Band (UWB), Bluetooth, WiMax, or any form of wireless communication protocol.

In some embodiments, volatile memory 932 includes, but is not limited to, Synchronous Dynamic Random Access Memory (SDRAM), Dynamic Random Access Memory (DRAM), RAMBUS Dynamic Random Access Memory (RDRAM), and/or any other type of random access memory device. Non-volatile memory 934 includes, but is not limited to, flash memory, phase change memory (PCM), read-only memory (ROM), electrically erasable programmable read-only memory (EEPROM), or any other type of non-volatile memory device.

Memory 930 stores information and instructions to be executed by processor 910. In one embodiment, memory 930 may also store temporary variables or other intermediate information while processor 910 is executing instructions. In the illustrated embodiment, chipset 920 connects with processor 910 via Point-to-Point (PtP or P-P) interfaces 917 and 922. Chipset 920 enables processor 910 to connect to other elements in system 900. In some embodiments of the invention, interfaces 917 and 922 operate in accordance with a PtP communication protocol such as the Intel® QuickPath Interconnect (QPI) or the like. In other embodiments, a different interconnect may be used.

In some embodiments, chipset 920 is operable to communicate with processor 910, 905N, display device 940, and other devices. Chipset 920 may also be coupled to a wireless antenna 978 to communicate with any device configured to transmit and/or receive wireless signals.

Chipset 920 connects to display device 940 via interface 926. Display 940 may be, for example, a liquid crystal display (LCD), a plasma display, cathode ray tube (CRT) display, or any other form of visual display device. In some embodiments of the invention, processor 910 and chipset 920 are merged into a single SOC. In addition, chipset 920 connects to one or more buses 950 and 955 that interconnect various elements 974, 960, 962, 964, and 966. Buses 950 and 955 may be interconnected together via a bus bridge 972. In one embodiment, chipset 920 couples with a non-volatile memory 960, a mass storage device(s) 962, a keyboard/mouse 964, and a network interface 966 via interface 924 and/or 904, etc.

In one embodiment, mass storage device 962 includes, but is not limited to, a solid state drive, a hard disk drive, a universal serial bus flash memory drive, or any other form of computer data storage medium. In one embodiment, network interface 966 is implemented by any type of well-known network interface standard including, but not limited to, an Ethernet interface, a universal serial bus (USB) interface, a Peripheral Component Interconnect (PCI) Express interface, a wireless interface and/or any other suitable type of interface. In one embodiment, the wireless interface operates in accordance with, but is not limited to, the IEEE 802.11 standard and its related family, Home Plug AV (HPAV), Ultra Wide Band (UWB), Bluetooth, WiMax, or any form of wireless communication protocol.

While the components shown in FIG. 9 are depicted as separate blocks within the system 900, the functions performed by some of these blocks may be integrated within a single semiconductor circuit or may be implemented using two or more separate integrated circuits. For example, although cache memory 916 is depicted as a separate block within processor 910, cache memory 916 (or selected aspects of 916) can be incorporated into processor core 912.

### Additional Notes and Examples

In Example 1 a system can include a first patch including a flexible, stretchable first substrate, a first adhesive on the first substrate, the first adhesive configured to attach the first patch to skin of a user, and first electronics on or at least partially in the first substrate, the first electronics to monitor a first biological parameter of the user, and a second patch including a flexible, stretchable second substrate, a second adhesive on the second substrate, the second adhesive configured to attach the second patch to skin of the user, and second electronics on or at least partially in the second substrate, the second electronics to monitor a second biological parameter of the user, the second biological parameter different from the first biological parameter.

In Example 2, Example 1 can further include, wherein the first biological parameter includes one of flexion, contraction, stretch, temperature, electrical activity a specific force, angular rate, magnetic field, salinity, muscular activity of the heart, electrical activity of the heart, pulse, oxygen content, lactate content, pressure, organ or conduit volume, blood pressure, and electro-dermal activity, and the second biological parameter includes one of one of flexion, contraction, stretch, temperature, electrical activity a specific force, angular rate, magnetic field, salinity, muscular activity of the heart, electrical activity of the heart, pulse, oxygen content, lactate content, pressure, organ or conduit volume, blood pressure, and electro-dermal activity.

In Example 3, at least one of Examples 1-2 can further include, wherein the first patch further includes a cavity in the first substrate, and wherein the first electronics are situated at least partially in the cavity.

In Example 4, Example 3 can further include, wherein the first adhesive is situated on the first substrate and the electronics are exposed to the skin when the first patch is worn.

In Example 5, Example 3 can further include, wherein the first adhesive is situated on the first substrate and the first electronics.

In Example 6, Example 1 can further include, wherein the first electronics are directly on the first substrate.

In Example 7, at least one of Examples 1-6 can further include, wherein the first patch is configured to be situated on a shoulder and the first electronics monitor one of flexion, contraction, stretch, temperature, electrical activity, specific force, angular rate, and magnetic field and wherein the second patch is configured to be situated on a bust and the second electronics monitor one of electrical activity of a heart, muscular activity of the heart, stretch, organ volume, conduit volume, blood pressure, temperature, respiration rate, salinity, oxygen, a specific force, angular rate, and magnetic field.

In Example 8, at least one of Examples 1-6 can further include, wherein the first patch is configured to be situated on a first knee and the first electronics monitor one of flexibility, stretch, temperature, electrical activity of the first knee, specific force, angular rate, and magnetic field and wherein the second patch is configured to be situated on a second knee and the second electronics monitor one of flexibility, stretch, temperature, electrical activity of the second knee, specific force, angular rate, and magnetic field.

In Example 9, at least one of Examples 1-6 can further include, wherein the first patch is configured to be situated on a neck and the first electronics monitor one of muscular activity of the heart, electrical activity of the heart, temperature, stretch, muscular pressure, blood pressure, organ volume, conduit volume, electrical activity, specific force, angular rate, and magnetic field and wherein the second patch is configured to be situated on a back and the second electronics monitor one of stretch, temperature, electrical activity of the back, specific force, angular rate, and magnetic field.

In Example 10, at least one of Examples 1-6 can further include, wherein the first patch is configured to be situated on a first elbow and the first electronics monitor one of flexibility, stretch, temperature, electrical activity, a specific force, angular rate, and magnetic field and wherein the second patch is configured to be situated on a wrist and the second electronics monitor one of stretch, temperature, electrical activity of the wrist, specific force, angular rate, and magnetic field.

In Example 11, at least one of Examples 1-10 can further include, wherein the first and second substrates include one of elastomer, spandex, woven fabric, polyvinyl chloride (PVC), polyethylene, polyurethane, thermoplastic polyethylene (TPU), polydimethylsiloxane (PDMS), latex, and a combination thereof.

In Example 12, at least one of Examples 1-11 can further include, wherein the first electronics include a flexible circuit board.

In Example 13, at least one of Examples 1-12 can further include, wherein the first electronics include a rigid circuit board.

In Example 14, at least one of Examples 1-13 can further include, wherein the first electronics include a feedback indicator including one of a speaker, a light or display, and a motor to provide feedback to the user, wherein the feedback includes activating the feedback indicator in response to a processor of the electronics determining that the biological parameter being monitored is out of a specified range of values for a specified period of time.

In Example 15, at least one of Examples 1-14 can further include, wherein the second electronics include a radio transmitter to provide signals indicative of the second biological parameter to a radio receiver of the first electronics.

In Example 16, at least one of Examples 1-15 can further include, wherein the first electronics include first processing circuitry, a first memory to store first data indicative of the first biological parameter, and a first connector electrical connector electrically connected to the processor, wherein an external device can retrieve first data from the first memory through the first processor providing the first data to the first connector, and the second electronics include second processing circuitry, a second memory to store second data indicative of the second biological parameter, and a second connector electrical connector electrically connected to the processor, wherein an external device can retrieve second data from the second memory through the second processor providing the second data to the second connector.

In Example 17, a method can further include attaching, using a first adhesive on a flexible, stretchable first substrate, a first patch to skin of a user at a first location, monitoring, using first electronics on or at least partially in the first substrate and during a time frame, a first biological parameter of the user, attaching, using a second adhesive on a flexible, stretchable second substrate, a second patch to the skin of the user at a second, different location, and monitoring, using second electronics on or at least partially in the second substrate, a second biological parameter of the user during the first time frame.

In Example 18, Example 17 can further include, wherein the first biological parameter includes one of flexion, contraction, stretch, temperature, electrical activity a specific force, angular rate, magnetic field, salinity, muscular activity of the heart, electrical activity of the heart, pulse, oxygen content, lactate content, pressure, organ or conduit volume, blood pressure, and electro-dermal activity, and the second biological parameter includes one of one of flexion, contraction, stretch, temperature, electrical activity a specific force, angular rate, magnetic field, salinity, muscular activity of the heart, electrical activity of the heart, pulse, oxygen content, lactate content, pressure, organ or conduit volume, blood pressure, and electro-dermal activity.

In Example 19, at least one of Examples 17-18 can further include, wherein the first location is a shoulder and the first biological parameter includes one of flexion, contraction, stretch, temperature, electrical activity, specific force, angular rate, and magnetic field and wherein the second location is a bust and the second biological parameter includes one of electrical activity of a hear, muscular activity of the heart, stretch, organ volume, conduit volume, blood pressure, temperature, respiration rate, salinity, oxygen, a specific force, angular rate, and magnetic field.

In Example 20, at least one of Examples 17-18 can further include, wherein the first location includes a first knee and the first biological parameter includes one of flexibility, stretch, temperature, electrical activity of the first knee, specific force, angular rate, and magnetic field and wherein the second location is a second knee and the second biological parameter includes one of flexibility, stretch, temperature, electrical activity of the second knee, specific force, angular rate, and magnetic field.

In Example 21, at least one of Examples 17-18 can further include, wherein the first location is a neck and the first biological parameter includes one of muscular activity of the heart, electrical activity of the heart, temperature, stretch, muscular pressure, blood pressure, organ volume, conduit volume, electrical activity, specific force, angular rate, and magnetic field and wherein the second location is a back and the second biological parameter includes one of stretch, temperature, electrical activity of the back, specific force, angular rate, and magnetic field.

In Example 22, at least one of Examples 17-18 can further include, wherein the first location is a first elbow and the first biological parameter includes one of flexibility, stretch, temperature, electrical activity, a specific force, angular rate, and magnetic field and wherein the second location is a wrist and the second biological parameter includes one of stretch, temperature, electrical activity of the wrist, specific force, angular rate, and magnetic field.

In Example 23, at least one of Examples 17-22 can further include, wherein the first and second substrates include one of elastomer, spandex, woven fabric, polyvinyl chloride (PVC), polyethylene, polyurethane, thermoplastic polyethylene (TPU), polydimethylsiloxane (PDMS), latex, and a combination thereof.

In Example 24, at least one of Examples 17-23 can further include, wherein the first electronics include a flexible circuit board.

In Example 25, at least one of Examples 17-24 can further include, wherein the first electronics include a rigid circuit board.

In Example 26, at least one of Examples 17-25 can further include monitoring, using processing circuitry of the first electronics, a first value of the first biological parameter, and in response to processing circuitry of the first electronics determining that the first biological parameter is out of a specified range of values for a specified period of time, providing, using a feedback indicator of the first electronics, feedback to the user including one of an audible, visual, or tactile signal using a speaker, a light or display, and a motor, respectively, of the first electronics.

In Example 27, at least one of Examples 17-26 can further include providing, using a radio transmitter of the second electronics, signals indicative of the second biological parameter to a radio receiver of the first electronics.

In Example 28, at least one of Examples 17-26 can further include, wherein the first electronics include first processing circuitry, a first memory to store first data indicative of the first biological parameter, and a first electrical connector electrically connected to the processor, and wherein the method further comprises providing, using the first electrical connector, first processing circuitry, and the first memory, an external device the first data in response to the first external device being electrically connected to the first connector, the second electronics include second processing circuitry, a second memory to store second data indicative of the second biological parameter, and a second electrical connector electrically connected to the second processing circuitry, and wherein the method further comprises providing, using the second electrical connector, second processing circuitry, and the second memory, the external device the second data in response to the first external device being electrically connected to the first connector.

Example 29 a system can include a first patch including a flexible, stretchable first substrate including a thermoplastic or thermoset elastomer on a stretchable fabric, a first adhesive on the first substrate, the first adhesive configured to attach the first patch to skin of a user, and first electronics on or at least partially in the first substrate, the first electronics to monitor electrocardiogram (ECG), pulse oximetry, and breathing of the user, the first electronics including first processing circuitry, a first memory to store data indicative of the first biological parameter electrically coupled to the first processing circuitry, and a second patch including a flexible, stretchable second substrate, a second adhesive on the second substrate, the second adhesive configured to attach the first patch to skin of the user, and second electronics on or at least partially in the second substrate, the second electronics to monitor posture of the user, the second biological parameter different from the first biological parameter.

The above description of embodiments includes references to the accompanying drawings, which form a part of the description of embodiments. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) can be used in combination with each other. Other embodiments can be used such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above description of embodiments, various features can be grouped together to streamline the disclosure. This should not be interpreted as intending that an unclaimed disclosed feature is essential to any claim. Rather, inventive subject matter can lie in less than all features of a particular disclosed embodiment. Thus, the following claims are hereby incorporated into the description of embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A system comprising:
a first patch including:
a flexible, stretchable first substrate,
a first adhesive on the first substrate, the first adhesive configured to attach the first patch to skin of a user, and
first electronics on or at least partially in the first substrate, the first electronics to monitor a first biological parameter of the user, and
a second patch including:
a flexible, stretchable second substrate,
a second adhesive on the second substrate, the second adhesive configured to attach the second patch to skin of the user, and
second electronics on or at least partially in the second substrate, the second electronics to monitor a second biological parameter of the user, the second biological parameter different from the first biological parameter.

2. The system of claim 1, wherein the first biological parameter includes one of flexion, contraction, stretch, temperature, electrical activity, a specific force, angular rate, magnetic field, salinity, muscular activity of the heart, electrical activity of the heart, pulse, oxygen content, lactate content, pressure, organ or conduit volume, blood pressure, and electro-dermal activity, and the second biological parameter includes one of one of flexion, contraction, stretch, temperature, electrical activity a specific force, angular rate, magnetic field, salinity, muscular activity of the heart, electrical activity of the heart, pulse, oxygen content, lactate content, pressure, organ or conduit volume, blood pressure, and electro-dermal activity.

3. The system of claim 1, wherein the first patch further includes a cavity in the first substrate, and wherein the first electronics are situated at least partially in the cavity.

4. The system of claim 3, wherein the first adhesive is situated on the first substrate and the electronics are exposed to the skin when the first patch is worn.

5. The system of claim 3, wherein the first adhesive is situated on the first substrate and the first electronics.

6. The system of claim 1, wherein the first electronics are directly on the first substrate.

7. The system of claim 1, wherein the first patch is configured to be situated on a shoulder and the first electronics monitor one of flexion, contraction, stretch, temperature, electrical activity, specific force, angular rate, and magnetic field and wherein the second patch is configured to be situated on a bust and the second electronics monitor one of electrical activity of a heart, muscular activity of the heart, stretch, organ volume, conduit volume, blood pressure, temperature, respiration rate, salinity, oxygen, a specific force, angular rate, and magnetic field.

8. The system of claim 1, wherein the first patch is configured to be situated on a first knee and the first electronics monitor one of flexibility, stretch, temperature, electrical activity of the first knee, specific force, angular rate, and magnetic field and wherein the second patch is configured to be situated on a second knee and the second electronics monitor one of flexibility, stretch, temperature, electrical activity of the second knee, specific force, angular rate, and magnetic field.

9. The system of claim 1, wherein the first patch is configured to be situated on a neck and the first electronics monitor one of muscular activity of the heart, electrical activity of the heart, temperature, stretch, muscular pressure, blood pressure, organ volume, conduit volume, electrical activity, specific force, angular rate, and magnetic field and wherein the second patch is configured to be situated on a back and the second electronics monitor one of stretch, temperature, electrical activity of the back, specific force, angular rate, and magnetic field.

10. The system of claim 1, wherein the first patch is configured to be situated on a first elbow and the first electronics monitor one of flexibility, stretch, temperature, electrical activity, a specific force, angular rate, and magnetic field and wherein the second patch is configured to be situated on a wrist and the second electronics monitor one of stretch, temperature, electrical activity of the wrist, specific force, angular rate, and magnetic field.

11. The system of claim 1, wherein the first and second substrates include one of elastomer, spandex, woven fabric, polyvinyl chloride (PVC), polyethylene, polyurethane, thermoplastic polyethylene (TPU), polydimethylsiloxane (PDMS), latex, and a combination thereof.

12. The system of claim 1, wherein the first electronics include a flexible circuit board.

13. The system of claim 1, wherein the first electronics include a rigid circuit board.

14. The system of claim 1, wherein the first electronics include a feedback indicator including one of a speaker, a light or display, and a motor to provide feedback to the user, wherein the feedback includes activating the feedback indicator in response to a processor of the electronics determining that the biological parameter being monitored is out of a specified range of values for a specified period of time.

15. The system of claim 1, wherein the second electronics include a radio transmitter to provide signals indicative of the second biological parameter to a radio receiver of the first electronics.

16. The system of claim 1, wherein:
the first electronics include:
first processing circuitry,
a first memory to store first data indicative of the first biological parameter, and
a first connector electrical connector electrically connected to the processor, wherein an external device can retrieve first data from the first memory through the first processor providing the first data to the first connector, and
the second electronics include:
second processing circuitry,
a second memory to store second data indicative of the second biological parameter, and
a second connector electrical connector electrically connected to the processor, wherein an external device can retrieve second data from the second memory through the second processor providing the second data to the second connector.

17. A method comprising:
attaching, using a first adhesive on a flexible, stretchable first substrate, a first patch to skin of a user at a first location;
monitoring, using first electronics on or at least partially in the first substrate and during a time frame, a first biological parameter of the user;
attaching, using a second adhesive on a flexible, stretchable second substrate, a second patch to the skin of the user at a second, different location; and
monitoring, using second electronics on or at least partially in the second substrate, a second biological parameter of the user during the first time frame.

18. The method of claim 17, wherein the first biological parameter includes one of flexion, contraction, stretch, temperature, electrical activity, a specific force, angular rate, magnetic field, salinity, muscular activity of the heart, electrical activity of the heart, pulse, oxygen content, lactate content, pressure, organ or conduit volume, blood pressure, and electro-dermal activity, and the second biological parameter includes one of one of flexion, contraction, stretch, temperature, electrical activity a specific force, angular rate, magnetic field, salinity, muscular activity of the heart, electrical activity of the heart, pulse, oxygen content, lactate content, pressure, organ or conduit volume, blood pressure, and electro-dermal activity.

19. The method of claim 17, wherein the first location is a shoulder and the first biological parameter includes one of flexion, contraction, stretch, temperature, electrical activity, specific force, angular rate, and magnetic field and wherein the second location is a bust and the second biological parameter includes one of electrical activity of a hear, muscular activity of the heart, stretch, organ volume, conduit volume, blood pressure, temperature, respiration rate, salinity, oxygen, a specific force, angular rate, and magnetic field.

20. A system comprising:
a first patch including:
a flexible, stretchable first substrate including a thermoplastic or thermoset elastomer on a stretchable fabric,
a first adhesive on the first substrate, the first adhesive configured to attach the first patch to skin of a user, and
first electronics on or at least partially in the first substrate, the first electronics to monitor electrocardiogram (ECG), pulse oximetry, and breathing of the user, the first electronics including first processing circuitry, a first memory to store data indicative of the first biological parameter electrically coupled to the first processing circuitry, and
a second patch including:
a flexible, stretchable second substrate,
a second adhesive on the second substrate, the second adhesive configured to attach the first patch to skin of the user, and
second electronics on or at least partially in the second substrate, the second electronics to monitor posture of the user, the second biological parameter different from the first biological parameter.
